(19) United States
(12) Patent Application Publication
(10) Pub. No.: US 2023/0320956 A1
(43) Pub. Date: Oct. 12, 2023

(54) DIARYLALKANE COMPOUNDS AND COMPOSITIONS FOR USE WITH SKIN AGING AND METHODS OF PRODUCTION THEREOF

(71) Applicant: Unigen, Inc., Tacoma, WA (US)

(72) Inventors: Teresa Horm, Tacoma, WA (US); Lidia Brownell, Tacoma, WA (US); Qi Jia, Olympia, WA (US)

(73) Assignee: Unigen, Inc., Tacoma, WA (US)

(21) Appl. No.: 18/133,294

(22) Filed: Apr. 11, 2023

Related U.S. Application Data
(60) Provisional application No. 63/330,002, filed on Apr. 12, 2022.

(51) Int. Cl. A61K 8/34; A61K 8/9794; A61Q 19/08

(52) U.S. Cl. CPC A61K 8/347; A61K 8/9794; A61Q 19/08; A61K 2800/522

(57) ABSTRACT

Diarylalkane compounds for use in anti-aging, wrinkle reduction, and extracellular matrix-boosting topical skincare are disclosed. Data and information from clinical trials that demonstrated reduced numbers of fine lines and wrinkles, reduced wrinkle depth, and reduced skin roughness upon treatment with the compound are also disclosed.

This United States Patent Application claims priority to U.S. Provisional Patent Application Ser. No. 63/330,002 filed on Apr. 12, 2022, which is commonly-owned and incorporated herein it its entirety.

### FIELD OF THE SUBJECT MATTER

Contemplated compounds and compositions relate to a topical application of at least one diarylalkane compound, including dimethoxytolyl propylresorcinol (as found in the International Nomenclature of Cosmetic Ingredients or INCI) for reversing or preventing the signs of skin aging, specifically fine lines, coarse lines, periorbital lines, and wrinkles. Specifically, contemplated compounds and compositions include active ingredients for the production and use of anti-aging skin care formulations.

### BACKGROUND

There is a need for skincare ingredients that reverse or prevent signs of skin aging, such as fine lines and wrinkles on facial skin, and especially on periorbital skin. Wrinkles form in the skin with aging due to a natural decline in skin cell replication, skin elasticity and water content, which can be accelerated by ultraviolet light exposure, smoking, and pollutant exposure (Farage M A, 2007) (Fore, 2006) (Haydont V, 2019). The skin is composed of the epidermis, which comprises several layers of squamous cells that form the external skin barrier, beneath which is the basement membrane (Michalak M, 2021). As with all basement membranes in epithelial tissues, it is the interface between the cell layer and the extracellular matrix. The basement membrane in skin is referred to as the dermal-epidermal junction, and as skin ages, the signaling between these compartments becomes impaired, as does the vascular support and structural support to the epidermis (Langton A K, 2016). The extracellular matrix beneath the basement membrane comprises the dermis layer of skin. This layer contains fibroblasts, nerves, blood vesicles, hair follicles, sweat glands, and sebaceous glands, as well as forming the matrix through which immune cells travel. The extracellular matrix is comprised of fibrillar proteins and water-binding glycosaminoglycans, the composition of which determines the cushioning and elasticity of the skin. Hyaluronic acid (HA) is a glycosaminoglycan that is naturally present in connective tissues throughout the body. It binds to cellular HA receptors, causing cell signaling cascades that affect the survival, proliferation, adhesion and migration activities of cells. In the skin, its main function is to bind to water molecules to retain moisture within the tissue and preserve the integrity of the dermis skin layer (Bukhari S N A, 2018). The fibrous proteins in the dermis include collagen, fibronectin, laminin, and elastin. These proteins are secreted by fibroblasts present in the ECM to form the structural component of the dermis of the skin. Downregulation of ECM proteins leads to thinning of the dermis layer and a reduction in water content, compromising structural support for the skin, resulting in skin sagging and visible wrinkles (Farage M A, 2007; Fore, 2006; Haydont V, 2019). Current topical treatments to reduce wrinkles include retinoids, vitamins, hydroxy acids, peptides, extracts and others. Their mechanisms of action include moisturizing the skin to increase water content, filling the skin to reduce the appearance of wrinkles, increasing skin cell turnover, increasing collagen production, and retaining collagen and other proteins in the dermis (Imhof L, 2021).

### SUMMARY OF THE SUBJECT MATTER

Diarylalkane compounds for use in anti-aging, wrinkle reduction, and extracellular matrix-boosting are contemplated and their uses are disclosed. Contemplated diarylalkane compounds include dimethoxytolyl propylresorcinol.

Contemplated compositions comprise the compound in an amount between 0.001-2 weight percent. In some embodiments, contemplated compositions comprise the compound in an amount of about 0.2 weight percent.

### BRIEF DESCRIPTION OF THE FIGURE

FIG. 1 shows a contemplated diarylalkane compound for use in anti-aging, wrinkle reduction, which is dimethoxytolyl propylresorcinol.

### DETAILED DESCRIPTION

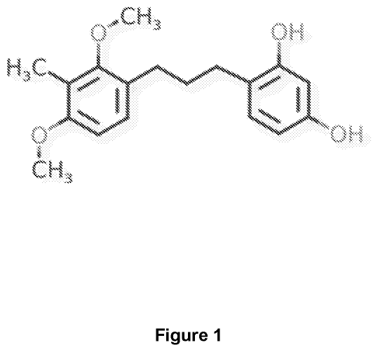

Contemplated diarylalkane compounds, including those with the INCI name as dimethoxytolyl propylresorcinol, as shown in FIG. 1, and with chemical name as 1-(3-methyl-2,4-dimethoxyphenyl)-3-(2′,4′-dihydroxyphenyl)-propane, or 1-(3-methyl-2,4-dimethoxyphenyl)-3-(2′,5′-dihydroxyphenyl)-propane, have previously been isolated from Dianella ensifolia and used for other skin care applications, not related to anti-aging or wrinkle treatment (Nesterov A, 2008). The extraction and purification method of the compound from Dianella ensifolia, and the organic synthesis method to produce the compound and usage for skin whitening based on tyrosinase inhibition were patented in U.S. Pat. Nos. 7,767,661; 8,592,488; 8,729,136; 9,126,913; and 10,857,082 B2.

Contemplated embodiments relate to the reversal and prevention of aging-related fine lines and wrinkles on skin. Specifically, contemplated compounds and compositions include at least one topical ingredient for reducing the number and appearance of fine lines and wrinkles in a clinical trial, increasing luminance in an Asian population, and decreasing blotchiness and age spots in a Caucasian population. The mechanism of action of contemplated embodiments was explored through in vitro gene expression and protein expression experiments, and the invention was found to increase proteins involved in the extracellular matrix. Additional gene expression changes observed included increases in genes relating to cytoskeleton, immune signaling, cell signaling, DNA repair, transcription, antioxidation, cell growth, skin barrier, wound healing, and keratinocyte differentiation.

Contemplated diarylalkane compounds and compositions including those compounds, including those with INCI name as dimethoxytolyl propylresorcinol, are highly effective for use as a topical anti-wrinkle ingredient in topical formulations, as evidenced by statistically significant reductions in the numbers of fine lines, skin roughness and wrinkle depth in female subjects in a clinical trial. There was also an increase in luminance/brightness in an Asian subject population, and a reduction in blotchiness and age spots in a Caucasian subject population.

These results, as are shown herein, were corroborated by in vitro increases in extracellular matrix genes and genes that increase hyaluronic acid synthesis. Additional genes that were found to be upregulated had such skin-related functions as wound healing, cytoskeletal regulation, antioxidation, immune signaling, cell growth, cell signaling, DNA repair, transcriptional regulation, and skin barrier function.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the terms “about” and “consisting essentially of” mean±20% of the indicated range, value, or structure, unless otherwise indicated. It should be understood that the terms “a” and “an” as used herein refer to “one or more” or “at least one” of the enumerated components. The use of the alternative (e.g., “and/or”) should be understood to mean either one, both, or any combination thereof of the alternatives. Unless the context requires otherwise, throughout the present specification and claims, the word “comprise” and variations thereof, such as, “comprises” and “comprising,” as well as synonymous terms like “include” and “have” and variants thereof, are to be construed in an open, inclusive sense; that is, as “including, but not limited to.”

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment.

The term “prodrug” is also meant to include any covalently bonded carriers, which release the active compound of this disclosure in vivo when such prodrug is administered to a mammalian subject. Prodrugs of a compound of this disclosure may be prepared by modifying functional groups present in the compound of this disclosure in such a way that the modifications are cleaved, either in routine manipulation or in vivo, to the parent compound of this disclosure. Prodrugs include compounds of this disclosure wherein a hydroxy, amino or mercapto group is bonded to any group that, when the prodrug of the compound of this disclosure is administered to a mammalian subject, cleaves to form a free hydroxy, free amino or free mercapto group, respectively. Examples of prodrugs include acetate, formate and benzoate derivatives of alcohol or amide derivatives of amine functional groups in the compounds of this disclosure and the like.

“Stable compound” and “stable structure” are meant to indicate a compound that is sufficiently robust to survive isolation to a useful degree of purity from a reaction mixture, and formulation into an efficacious therapeutic agent.

“Biomarker(s)” or “marker(s)” component(s) or compound(s) are meant to indicate one or multiple indigenous chemical component(s) or compound(s) in the disclosed plant(s), plant extract(s), or combined composition(s) with 2-3 plant extracts that are utilized for controlling the quality, consistence, integrity, stability, and/or biological functions of the invented composition(s).

“Mammal” includes humans and both domestic animals, such as laboratory animals or household pets (e.g., cats, dogs, swine, cattle, sheep, goats, horses, rabbits), and non-domestic animals, such as wildlife or the like.

“Optional” or “optionally” means that the subsequently described element, component, event or circumstances may or may not occur, and that the description includes instances where the element, component, event or circumstance occur and instances in which they do not. For example, “optionally substituted aryl” means that the aryl radical may or may not be substituted and that the description includes both substituted aryl radicals and aryl radicals having no substitution.

“Pharmaceutically or nutraceutically acceptable carrier, diluent or excipient” includes any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically or nutraceutically acceptable salt” includes both acid and base addition salts. “Pharmaceutically or nutraceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid and the like, and organic acids such as acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically or nutraceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include the sodium, potassium, lithium, ammonium, calcium, magnesium, iron, zinc, copper, manganese, aluminum salts and the like. In certain embodiments, the inorganic salts are ammonium, sodium, potassium, calcium, or magnesium salts. Salts derived from organic bases include salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2 dimethylaminoethanol, 2 diethylaminoethanol, dicyclohexylamine, lysine, arginine, histidine, procaine, hydrabamine, choline, betaine, benethamine, benzathine, ethylenediamine, glucosamine, methylglucamine, theobromine, triethanolamine, tromethamine, purines, piperazine, piperidine, N ethylpiperidine, polyamine resins and the like. Particularly useful organic bases are isopropylamine, diethylamine, ethanolamine, trimethylamine, dicyclohexylamine, choline and caffeine.

Often crystallizations produce a solvate of the compound of this disclosure. As used herein, the term “solvate” refers to an aggregate that comprises one or more molecules of a compound of this disclosure with one or more molecules of solvent. The solvent may be water, in which case the solvate may be a hydrate. Alternatively, the solvent may be an organic solvent. Thus, the compounds of the present invention may exist as a hydrate, including a monohydrate, dihydrate, hemihydrate, sesquihydrate, trihydrate, tetrahydrate and the like, as well as the corresponding solvated forms. The compound of this disclosure may be true solvates, while in other cases, the compound of this disclosure may merely retain adventitious water or be a mixture of water plus some adventitious solvent.

A “pharmaceutical composition” or “nutraceutical composition” refers to a formulation of a compound of this disclosure and a medium generally accepted in the art for the delivery of the biologically active compound to mammals, e.g., humans. For example, a pharmaceutical composition of the present disclosure may be formulated or used as a standalone composition, or as a component in a prescription drug, an over the counter (OTC) medicine, a botanical drug, an herbal medicine, a natural medicine, a homeopathic agent, or any other form of health care product reviewed and approved by a government agency. Exemplary nutraceutical compositions of the present disclosure may be formulated or used as a standalone composition, or as a nutritional or bioactive component in food, a functional food, a beverage, a bar, a food flavor, a medical food, a dietary supplement, or an herbal product. A medium generally accepted in the art includes all pharmaceutically or nutraceutically acceptable carriers, diluents or excipients therefor.

As used herein, “enriched for” refers to a plant extract or other preparation having at least a two-fold up to about a 1000-fold increase of one or more active compounds as compared to the amount of one or more active compounds found in the weight of the plant material or other source before extraction or other preparation. In certain embodiments, the weight of the plant material or other source before extraction or other preparation may be dry weight, wet weight, or a combination thereof.

As used herein, “major active ingredient” or “major active component” refers to one or more active compounds found in a plant extract or other preparation or enriched for in a plant extract or other preparation, which is capable of at least one biological activity. In certain embodiments, a major active ingredient of an enriched extract will be the one or more active compounds that were enriched in that extract. Generally, one or more major active components will impart, directly or indirectly, most (i.e., greater than 50%, or 20% or 10%) of one or more measurable biological activities or effects as compared to other extract components. In certain embodiments, a major active ingredient may be a minor component by weight percentage of an extract (e.g., less than 50%, 25%, or 10% or 5% or 1% of the components contained in an extract) but still provide most of the desired biological activity. Any composition of this disclosure containing a major active ingredient may also contain minor active ingredients that may or may not contribute to the pharmaceutical or nutraceutical activity of the enriched composition, but not to the level of major active components, and minor active components alone may not be effective in the absence of a major active ingredient.

“Effective amount” or “therapeutically effective amount” refers to that amount of a compound or composition of this disclosure which, when administered, is enough to reduce the appearance of wrinkles and fine lines, liver spots (solar lentigines), and other markers of skin aging.

The amount of a compound, an extract or a composition of this disclosure that constitutes a “therapeutically effective amount” will vary depending on the bioactive compound, or the biomarker for the condition being treated and its severity, the manner of administration, the duration of treatment, or the age of the subject to be treated, but can be determined routinely by one of ordinary skill in the art having regard to his own knowledge and to this disclosure.

“Treating” or “treatment” as used herein refers to the treatment of the disease or condition of interest in a mammal, such as a human, having the disease or condition of interest, and includes: (i) preventing the disease or condition from occurring in a mammal, in particular, when such mammal is predisposed to the condition but has not yet been diagnosed as having it; (ii) inhibiting the disease or condition, i.e., arresting its development; (iii) relieving or modifying the disease or condition, i.e., causing regression of the disease or condition; or (iv) relieving the symptoms resulting from the disease or condition, (e.g., reducing the appearance of fine lines, wrinkles, and liver spots (lentigines) without addressing the underlying disease or condition; (v) or changing the phenotype of the disease or condition.

As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

As used herein, “statistical significance” refers to a p value of 0.050 or less when calculated using the Student t-test and indicates that it is unlikely that a particular event or result being measured has arisen by chance.

For the purposes of administration, contemplated compounds, such as diarylalkane compounds including dimethoxytolyl propylresorcinol, may be administered as a raw chemical or may be formulated as pharmaceutical or nutraceutical compositions. Contemplated pharmaceutical or nutraceutical compositions comprise a compound of structures described herein and a pharmaceutically or nutraceutically acceptable carrier, diluent or excipient. The compound of structures described here are present in the composition in an amount which is effective to treat a particular disease or condition of interest—that is, in an amount sufficient to reduce the appearance of fine lines, wrinkles, and liver spot (solar lentigines) in general or any of the other associated indications described herein, and generally with acceptable toxicity to a patient.

Administration of contemplated diarylalkane compounds, including dimethoxytolyl propylresorcinol or other related compositions disclosed herein, or their pharmaceutically or nutraceutically acceptable salts, in pure form or in an appropriate pharmaceutical or nutraceutical composition, can be carried out via any of the accepted modes of administration of agents for serving similar utilities. The pharmaceutical or nutraceutical compositions of this disclosure can be prepared by combining a compound of this disclosure with an appropriate pharmaceutically or nutraceutically acceptable carrier, diluent or excipient, and may be formulated into preparations in solid, semi solid, liquid or gaseous forms, such as powders, granules, ointments, solutions, injections, inhalants, gels, creams, lotions, tinctures, masks, and microspheres. Typical routes of administering such pharmaceutical or nutraceutical compositions include oral, topical, transdermal, inhalation, parenteral, sublingual, buccal, rectal, vaginal, or intranasal. The term parenteral as used herein includes subcutaneous injections, intravenous, intramuscular, intrasternal injection or infusion techniques.

Pharmaceutical, dermatological or nutraceutical compositions disclosed herein are formulated so as to allow the active ingredients contained therein to be bioavailable upon administration of the composition to a patient. Compositions that will be administered to a subject or patient or a mammal take the form of one or more dosage units, where for example, a tablet may be a single dosage unit, and a container of a compound or an extract or a composition of 2-3 plant extracts of this disclosure in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a therapeutically effective amount of a compound of this disclosure, or a pharmaceutically or nutraceutically acceptable salt thereof, for treatment of a disease or condition of interest in accordance with the teachings disclosed herein.

A dermatological, pharmaceutical or nutraceutical composition of this disclosure may be in the form of a solid or liquid. In one aspect, the carrier(s) are particulate, so that the compositions are, for example, in tablet or in powder form. The carrier(s) may be liquid, with the compositions being, for example, oral syrup, injectable liquid or an aerosol, which is useful in, for example, inhalatory administration.

When intended for oral administration, the dermatological, pharmaceutical or nutraceutical composition is in either solid or liquid form, where semi solid, semi liquid, suspension and gel forms are included within the forms considered herein as either solid or liquid.

As a solid composition for oral administration, the dermatological, pharmaceutical or nutraceutical composition may be formulated into a powder, granule, compressed tablet, pill, capsule, chewing gum, sashay, wafer, bar, or like form. Such a solid composition will typically contain one or more inert diluents or edible carriers. In addition, one or more of the following may be present: binders such as carboxymethylcellulose, ethyl cellulose, cyclodextrin, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; and a coloring agent.

The liquid dermatological, pharmaceutical or nutraceutical compositions of this disclosure, whether they be solutions, suspensions or other like form, may include one or more of the following adjuvants: sterile diluents such as water for injection, saline solution, such as physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a generally useful adjuvant. An injectable pharmaceutical or nutraceutical composition is sterile.

The dermatological, pharmaceutical or nutraceutical composition of this disclosure may be intended for topical administration, in which case the carrier may suitably comprise a solution, emulsion, cream, lotion, ointment, or gel base. The base, for example, may comprise one or more of the following: petrolatum, lanolin, polyethylene glycols, bees wax, mineral oil, diluents such as water, alcohol, emulsifiers and stabilizers. Thickening agents may be present in a pharmaceutical or nutraceutical composition for topical administration. If intended for transdermal administration, the composition may include a transdermal patch or iontophoresis device.

The dermatological pharmaceutical or nutraceutical composition of this disclosure in solid or liquid form may include an agent that binds to the compound of this disclosure and thereby assists in the delivery of the compound. Suitable agents that may act in this capacity include a monoclonal or polyclonal antibody, a protein or a liposome.

The dermatological, pharmaceutical or nutraceutical composition of this disclosure in solid or liquid form may include reducing the size of a particle to, for example, improve bioavailability. The size of a powder, granule, particle, microsphere, or the like in a composition, with or without an excipient, can be macro (e.g., visible to the eye or at least 100 μm in size), micro (e.g., may range from about 100 μm to about 100 nm in size), nano (e.g., may no more than 100 nm in size), and any size in between or any combination thereof to improve size and bulk density.

The dermatological, pharmaceutical or nutraceutical compositions of this disclosure may be prepared by methodology well known in the pharmaceutical or nutraceutical art. For example, a pharmaceutical or nutraceutical composition intended to be administered by injection can be prepared by combining a compound of this disclosure with sterile, distilled, deionized water so as to form a solution. A surfactant may be added to facilitate the formation of a homogeneous solution or suspension. Surfactants are compounds that non covalently interact with the compound of this disclosure so as to facilitate dissolution or homogeneous suspension of the compound in the aqueous delivery system.

The compounds of this disclosure, or their dermatological, pharmaceutically or nutraceutically acceptable salts, are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy.

It is understood that in the present description, combinations of substituents or variables of the depicted formulae are permissible only if such contributions result in stable compounds.

It will also be appreciated by those skilled in the art that in the process described herein the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include C(O) R″ (where R″ is alkyl, aryl or arylalkyl), p methoxybenzyl, trityl and the like. Suitable protecting groups for carboxylic acid include alkyl, aryl or arylalkyl esters. Protecting groups may be added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin or a 2-chlorotrityl-chloride resin.

It will also be appreciated by those skilled in the art, although such protected derivatives of contemplated compounds disclosed herein may not possess dermatological and pharmacological activity as such, they may be administered to a mammal and thereafter metabolized in the body to form compounds of this disclosure which are pharmacologically active. Such derivatives may therefore be described as “prodrugs”. All prodrugs of compounds of this invention are included within the scope of this disclosure.

Furthermore, all compounds or extracts of this disclosure which exist in free base or acid form can be converted to their pharmaceutically or nutraceutically acceptable salts by treatment with the appropriate inorganic or organic base or acid by methods known to one skilled in the art. Salts of the compounds of this disclosure can be converted to their free base or acid form by standard techniques.

Contemplated compounds, medicinal compositions and compositions may comprise or additionally comprise or consist of at least one active ingredient. In some embodiments, at least one bioactive ingredient may comprise or consist of plant powder or plant extract of or the like.

Also contemplated herein are agents of the disclosed and contemplated diarylalkane compounds, including dimethoxytolyl propylresorcinol. Such products may result from, for example, the oxidation, reduction, hydrolysis, amidation, esterification, and the like of the administered compound, primarily due to enzymatic processes. Accordingly, contemplated compounds are those produced by a process comprising administering a contemplated compound or composition to a mammal for a period of time sufficient to yield a metabolic product thereof.

Contemplated compounds, medicinal compositions and compositions may comprise or additionally comprise or consist of at least one pharmaceutically or nutraceutically or cosmetically acceptable carrier, diluent or excipient. As used herein, the phrase “pharmaceutically or nutraceutically or cosmetically acceptable carrier, diluent or excipient” includes any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals. Contemplated compounds, medicinal compositions and compositions may comprise or additionally comprise or consist of at least one pharmaceutically or nutraceutically or cosmetically acceptable salt. As used herein, the phrase “pharmaceutically or nutraceutically or cosmetically acceptable salt” includes both acid addition and base addition salts.

In some embodiments, contemplated diarylalkane compounds, including those that include dimethoxytolyl propylresorcinol, can be isolated from plant and/or marine sources. Suitable plant parts for isolation of the compounds include leaves, bark, trunk, trunk bark, stem, stem bark, twigs, tubers, root, rhizome, root bark, bark surface, young shoots, seed, fruit, androecium, gynoecium, calyx, stamen, petal, sepal, carpel (pistil), flower, or any combination thereof. In some related embodiments, the compounds or extracts are isolated from plant sources and synthetically modified to contain any of the recited substituents. In this regard, synthetic modification of the compound isolated from plants can be accomplished using any number of techniques which are known in the art and are well within the knowledge of one of ordinary skill in the art. In some related embodiments, contemplated diarylalkane compounds, including those that include dimethoxytolyl propylresorcinol, are biosynthesized from plant tissues or fungi tissues, stem cells and transgenic microbials and synthetically modified by isolated or expressed enzymes to contain any of the recited substituents. In this regard, biosynthetic modification of contemplated compounds can be accomplished using any number of synthetic biology techniques which are known in the art and are well within the knowledge of one of ordinary skill in the art.

Herein, the anti-aging effects of contemplated diarylalkane compounds are disclosed, including those that include dimethoxytolyl propylresorcinol, on human keratinocytes in vitro and in human clinical trials. In keratinocytes treated with contemplated diarylalkane compounds, including those that include dimethoxytolyl propylresorcinol, we found profound similarities in differentially expressed genes compared to retinol-treated keratinocytes. Additional changes specific to contemplated diarylalkane compounds, including those that include dimethoxytolyl propylresorcinol, included increased extracellular matrix protein and proteoglycan synthesis and contributed to reorganization of the extracellular matrix. These activities and others enriched contemplated diarylalkane compounds, including those that include dimethoxytolyl propylresorcinol-treated keratinocytes, such as growth factor signaling, keratinocyte differentiation, cell-cell communication and cell-ECM communication are implicated in anti-aging. In a human clinical trial, we found that contemplated diarylalkane compounds, including those that include dimethoxytolyl propylresorcinol, in a 0.2% cream used twice daily on the face improved the appearance of fine lines and wrinkles, along with increasing skin brightness/luminance in an Asian population of human subjects and reducing skin blotchiness/age spots in a Caucasian population.

### EXAMPLES

The Examples herein focus on the use of dimethoxytolyl propylresorcinol, but it should be understood that similar diarylalkane compounds can be utilized and one of ordinary skill in the art will understand how those similar diarylalkane compounds can be utilized in compositions contemplated herein.

### Example 1. Global Gene Expression Changes in Keratinocytes Treated with Dimethoxytolyl Propylresorcinol

To determine specific expression changes dimethoxytolyl propylresorcinol exerted on human keratinocyte aging genes, keratinocytes (Hacat cell line) were treated dimethoxytolyl propylresorcinol at 40 μM and gene expression was analyzed by RNA-seq. Differential gene expression was compared to untreated keratinocytes and cells treated with 40 μM Retinol A to find genes involved in aging. Genes that were statistically significantly differentially regulated were included in the analysis (p<0.05). Retinol was chosen as a positive control because retinoids have been described as having effective anti-aging and anti-wrinkle effects (Zasada M, 2019). We directly compared the effects of Retinol on keratinocytes with the same concentration of dimethoxytolyl propylresorcinol.

### Protein Localizations And Biological Processes Enriched in Both Retinol and Dimethoxytolyl propylresorcinol-Treated Keratinocytes

First, we compared enriched biological processes that were common to both Retinol and dimethoxytolyl propylresorcinol-treated keratinocytes (Table 1). Protein localizations and biological processes related to skin aging were enriched in both Retinol and dimethoxytolyl propylresorcinol-treated keratinocytes, including Extracellular space, Proteinaceous extracellular matrix, Extracellular region, Epidermis development, and Epithelial cell differentiation.

TABLE 1
Protein localizations and biological processes upregulated in both
Retinol and dimethoxytolyl propylresorcinol-treated keratinocytes (listed
according to the number of genes affected in Retinol-treated keratinocytes)

| | | | Number of Genes | |
|---|---|---|---|---|
| | Number of Genes | | Differentially | |
| | Differentially | | Expressed in | Fold Enrichment |
| | Expressed in | Fold Enrichment | dimethoxytolyl | in dimethoxytolyl |
| Gene Cluster | Retinol | in Retinol | propylresorcinol | propylresorcinol |
| Extracellular exosome | 83 | 1.47 | 183 | 1.23 |
| Extracellular space | 67 | 2.48 | 140 | 1.97 |
| Extracellular region | 56 | 1.73 | 136 | 1.60 |
| Calcium ion binding | 30 | 2.04 | 58 | 1.49 |
| Proteinaceous | 23 | 4.27 | 35 | 2.47 |
| extracellular matrix | | | | |
| Inflammatory response | 20 | 2.54 | 54 | 2.61 |
| Cell surface | 20 | 1.84 | 53 | 1.85 |
| Negative regulation of | 20 | 2.43 | 47 | 2.17 |
| cell proliferation | | | | |
| Positive regulation of | 18 | 1.86 | 50 | 1.96 |
| cell proliferation | | | | |
| Heparin binding | 16 | 4.86 | 22 | 2.53 |
| Response to drug | 15 | 2.37 | 34 | 2.04 |
| Cell-cell signaling | 13 | 2.46 | 36 | 2.59 |
| Positive regulation of | 13 | 3.40 | 29 | 2.88 |
| cell migration | | | | |
| Extracellular matrix | 13 | 3.19 | 22 | 2.05 |
| organization | | | | |
| Positive regulation of | 12 | 2.20 | 26 | 1.82 |
| gene expression | | | | |
| Angiogenesis | 11 | 2.37 | 34 | 2.79 |
| Growth factor activity | 10 | 3.00 | 32 | 3.63 |
| Cellular response to | 10 | 4.26 | 19 | 3.08 |
| lipopolysaccharide | | | | |
| Epidermis | 8 | 4.53 | 21 | 4.52 |
| development | | | | |
| Positive regulation of | 8 | 3.35 | 19 | 3.02 |
| angiogenesis | | | | |
| Negative regulation of | 7 | 2.78 | 16 | 2.42 |
| endopeptidase activity | | | | |
| Osteoblast | 7 | 3.24 | 15 | 2.64 |
| differentiation | | | | |
| Activation of MAPK | 7 | 3.15 | 14 | 2.39 |
| activity | | | | |
| Negative regulation of | 7 | 5.81 | 11 | 3.47 |
| ERK1 and ERK2 | | | | |
| cascade | | | | |
| Epithelial cell | 7 | 4.81 | 11 | 2.87 |
| differentiation | | | | |
| Positive regulation of | 6 | 4.81 | 16 | 4.88 |
| smooth muscle cell | | | | |
| proliferation | | | | |
| Cellular response to | 6 | 4.12 | 12 | 3.14 |
| retinoic acid | | | | |
| Neutrophil chemotaxis | 5 | 3.65 | 12 | 3.33 |
| Transforming growth | 5 | 5.79 | 11 | 4.82 |
| factor beta receptor | | | | |
| binding | | | | |
| O-glycan processing | 5 | 4.00 | 10 | 3.05 |
| Cell development | 4 | 4.81 | 9 | 4.12 |
| Growth | 4 | 6.87 | 8 | 5.23 |
| Intermediate filament | 3 | 9.62 | 6 | 7.32 |
| cytoskeleton | | | | |
| organization | | | | |

### Protein Localizations and Biological Processes Enriched Only in Dimethoxytolyl Propyl Resorcinol-Treated Keratinocytes

We found several aging-related biological processes that were enriched in dimethoxytolyl propylresorcinol-treated keratinocytes, but not Retinol-treated keratinocytes in our study. Importantly, wound healing process enrichment indicates increased dynamics in the tissue, which may impact the appearance of aging phenotypes in the skin. Increased keratinocyte differentiation enrichment indicates enforcement of the skin barrier, which makes the skin less susceptible to extrinsic damage (Gutowska-Owsiak D, 2020). Hyaluronan is important for hydration of the skin, as it is a proteoglycan that binds water molecules in the extracellular matrix of the skin. The enriched processes in dimethoxytolyl propylresorcinol-treated keratinocytes, but not in Retinol-treated keratinocytes, are listed in Table 2.

TABLE 2
Gene Clusters enriched in dimethoxytolyl propylresorcinol-treated
keratinocytes (listed according to the number of genes affected in
dimethoxytolyl propylresorcinol-treated keratinocytes)

| | Number of | Fold |
|---|---|---|
| | Genes | Enrichment in |
| | Differentially | Diarylalkane |
| | Expressed in | compound with |
| | dimethoxytolyl | dimethoxytolyl |
| Gene Cluster | propylresorcinol | propylresorcinol |
| Wound healing | 14 | 3.20 |
| Keratinocyte differentiation | 13 | 3.13 |
| Hyaluronan biosynthetic | 4 | 10.45 |
| process | | |

### Genes Differentially Regulated in Both Retinol and Dimethoxytolyl Propylresorcinol-Treated Keratinocytes

Specific aging genes commonly regulated between Retinol and dimethoxytolyl propylresorcinol-treated keratinocytes are listed in Tables 3-8.
Extracellular matrix (ECM) genes that were upregulated in both Retinol and dimethoxytolyl propylresorcinol-treated keratinocytes included TFP12, which codes for a serine protease inhibitor that inhibits trypsin and matrix metalloproteases that break down the extracellular matrix, and CRISPLD2, which is thought to be involved in glycosaminoglycan binding and directly affect the integrity of the extracellular matrix. CEACAM1 is an immunoglobulin involved in cell adhesion and tissue structure, and ABCA12 is a transporter and integral membrane protein. Increased expression of these genes may indicate increased cell-ECM communication and adhesion.

Among ECM genes downregulated in both Retinol and dimethoxytolyl propylresorcinol-treated keratinocytes, all are involved in cell adhesion and are proteins and proteoglycans present in the extracellular matrix. The downregulation of these may be related to a change of the matrix composition, or changes in cell adhesion dynamics. Differential expression of these genes is listed in Table 3.

TABLE 3
ECM genes differentially expressed in both Retinol and dimethoxytolyl propylresorcinol -
treated keratinocytes (listed according to fold change in Retinol-treated keratinocytes)

| | | | Fold change in |
|---|---|---|---|
| | | Fold change in | dimethoxytolyl |
| Gene Cluster | Gene | Retinol | propylresorcinol |
| Upregulated | | | |
| ECM | TFP12 - Tissue factor pathway inhibitor 2 | 2.46 | 2.37 |
| ECM | CRISPLD2 - Cysteine-rich secretory | 1.98 | 2.56 |
| | protein LCCL domain-containing 2 | | |
| ECM | HS3ST1 - Heparan sulfate-glucosamine | 1.48 | 2.28 |
| | 3-sulfotransferase 1 | | |
| Cell adhesion | CEACAM1 - CEA cell adhesion molecule | 3.00 | 2.01 |
| | 1 | | |
| Downregulated | | | |
| ECM | FLRT2 - Fibronectin leucine-rich | 0.58 | 0.30 |
| | transmembrane protein 2 | | |
| Cell adhesion, | EPHA4 - Ephrin type-A receptor 4 | 0.55 | 0.42 |
| cell | | | |
| communication | | | |
| ECM | PSG5 - Pregnancy specific beta-1- | 0.37 | 0.30 |
| | glycoprotein 5 | | |
| ECM | CD1D - CD1d molecule | 0.32 | 0.36 |
| ECM | PSAPL1 - Prosaposin-like 1 | 0.32 | 0.30 |
| ECM | OLFM4 - Olfactomedin 4 | 0.28 | 0.10 |
| ECM | COL5A1 - Collagen type V alpha 1 chain | 0.27 | 0.24 |
| ECM | VIT - Vitrin | 0.14 | 0.08 |

Among upregulated genes involved in plasma membrane fusion, cytoskeleton remodeling, and lipid metabolism, RAPGEF3 activates small GTP-ases involved in cytoskeleton remodeling and plasma membrane dynamics, suggesting that Retinol and dimethoxytolyl propylresorcinol treatment affected cell-ECM interactions in keratinocytes. UGCG is involved in biosynthesis of membrane lipids. Upregulation may indicate increased cell-ECM communication and/or lipid turnover rate.
KIF13A, the microtubule motor protein, was downregulated in both Retinol and dimethoxytolyl propylresorcinol-treated keratinocytes. KIF13A is involved in endosome transport along microtubules, and its downregulation may indicate a change in trafficking dynamics. Differential expression of these genes is listed in Table 4.

TABLE 4
Cytoskeleton and transport genes differentially regulated in both
Retinol and dimethoxytolyl propylresorcinol-treated keratinocytes

| | | | Fold change in |
|---|---|---|---|
| | | Fold change in | dimethoxytolyl |
| Gene Cluster | Gene | Retinol | propylresorcinol |
| Upregulated | | | |
| Plasma membrane | RAPGEF3 - Rap guanine nucleotide | 1.81 | 1.78 |
| fusion/cytoskeleton | exchange factor 3 | | |
| Lipid metabolism | UGCG - UDP-glucose ceramide | 1.24 | 1.56 |
| and transport | glucosyltransferase | | |
| Downregulated | | | |
| Cytoskeleton/ | KIF13A - Kinesin family member 13A | 0.68 | 0.67 |
| secretion | | | |

ANOS1, TNC and SEMA3D are extracellular matrix proteins that are primarily involved in neural cell adhesion and axon elongation, so downregulation of these genes may indicate a shift away from innervation of the skin. Tenascin C (TNC) has been shown previously to be downregulated in retinoic acid-treated rat glioma cells (Alvarez-Dolado M, 1999). There may be other functions of these proteins in the extracellular matrix that affect skin aging and explain their downregulation in Retinol and dimethoxytolyl propylresorcinol-treated keratinocytes. Differential expression of these genes is listed in Table 5.

TABLE 5
Extracellular matrix proteins involved in neural cell adhesion
and axon elongation differentially expressed in both Retinol
and dimethoxytolyl propylresorcinol-treated keratinocytes

| | | | Fold change in |
|---|---|---|---|
| Gene Cluster | | Fold change in | dimethoxytolyl |
| Downregulated | Gene | Retinol | propylresorcinol |
| ECM/Nerve | ANOS1 - Anosmin 1 | 0.55 | 0.49 |
| adhesion | | | |
| ECM/Nerve | SEMA3D - Semaphorin 3D | 0.50 | 0.54 |
| growth | | | |
| ECM/Nerve | TNC - Tenascin C | 0.14 | 0.50 |
| growth | | | |

Both Retinol and dimethoxytolyl propylresorcinol-induced changes in the expression of growth-related genes. Generally, the net effect of these changes was a decrease in cell proliferation, as three genes involved in Negative regulation of growth were upregulated: PTPRR, C3orf33, and ANGPTL4, and four genes involved in Growth were downregulated: BMP4, FGF1, FGF11 and TP73. Differential expression of these genes is listed in Table 6.

TABLE 6
Growth genes differentially expressed in Retinol and
dimethoxytolyl propylresorcinol-treated keratinocytes

| | | | Fold change in |
|---|---|---|---|
| | | Fold change in | dimethoxytolyl |
| Gene Cluster | Gene | Retinol | propylresorcinol |
| Upregulated | | | |
| Negative | PTPRR - Protein tyrosine phosphatase | 2.31 | 3.96 |
| regulation of | receptor type R | | |
| growth | | | |
| Negative | C3orf33 - Chromosome 3 open reading | 2.24 | 3.03 |
| regulation of | frame 33 | | |
| growth | | | |
| Negative | ANGPTL4 - Angiopoietin-like 4 | 2.77 | 8.23 |
| regulation of | | | |
| growth/ | | | |
| Angiogenesis | | | |
| Downregulated | | | |
| Growth | BMP4 - Bone morphogenetic protein 4 | 0.51 | 0.41 |
| Growth | FGF1 - Fibroblast growth factor 1 | 0.45 | 0.43 |
| Growth | FGF11 - Fibroblast growth factor 11 | 0.50 | 0.56 |
| Growth | TP73 - Tumor protein P73 | 0.38 | 0.35 |

Immune signaling was increased in Retinol and dimethoxytolyl propylresorcinol-treated keratinocytes, as was CYP1A1, a cytochrome p450 family member oxygenase that is involved in drug metabolism. These genes may be upregulated in direct response to drug treatment, especially CYP1A1. The changes seen in immune signaling genes may demonstrate a role for increased immune signaling in Retinol and dimethoxytolyl propylresorcinol-treated keratinocytes. Differential expression of these genes is listed in Table 7.

TABLE 7
Immune genes differentially expressed in both Retinol and
dimethoxytolyl propylresorcinol-treated keratinocytes

| | | | Fold change in |
|---|---|---|---|
| Gene Cluster | | Fold change in | dimethoxytolyl |
| Upregulated | Gene | Retinol | propylresorcinol |
| Immune | CXCL3 - C-X-C motif chemokine ligand 3 | 2.60 | 8.16 |
| signaling | | | |
| Immune | IL1RL1 - Interleukin 1 receptor-like 1 | 3.13 | 17.29 |
| signaling | | | |
| Immune | TLR4 - Toll-like receptor 4 | 2.67 | 3.41 |
| signaling | | | |
| Drug metabolism | CYP1A1 - Cytochrome P450 family 1 | 2.30 | 5.04 |
| | subfamily A member 1 | | |

TXNRD1, an antioxidant enzyme, was upregulated in Retinol and dimethoxytolyl propylresorcinol-treated keratinocytes, increasing antioxidation capacity in the cells. Antioxidants are important for reducing photo-induced skin damage (Masaki, 2010). Differential expression of this gene is listed in Table 8.

TABLE 8
Antioxidation genes differentially expressed in both Retinol
and dimethoxytolyl propylresorcinol-treated keratinocytes

| | | | Fold change in |
|---|---|---|---|
| Gene Cluster | | Fold change in | dimethoxytolyl |
| Upregulated | Gene | Retinol | propylresorcinol |
| Oxidative stress | TXNRD1 - Thioredoxin reductase 1 | 1.45 | 1.52 |

### Genes Differentially Expressed Only in Dimethoxytolyl Propylresorcinol-Treated Keratinocytes

There was a large subset of skin aging genes that were differentially expressed in dimethoxytolyl propylresorcinol-treated keratinocytes, but not in Retinol-treated keratinocytes, listed in Tables 9-14.
Among Extracellular matrix and Cell adhesion genes, the hyaluronan synthases HAS2 and HAS3 were among the most highly upregulated in dimethoxytolyl propylresorcinol-treated keratinocytes. These genes are responsible for producing hyaluronan, a proteoglycan that is abundant in the extracellular matrix, and which is vital for skin hydration. LAMC2 and LAMA3 laminins, proteins in the extracellular matrix, were upregulated, as was COL1A1, the most abundant collagen in skin connective tissue. ECM1, an extracellular matrix protein, and NID2, an extracellular glycoprotein were also upregulated. PI3, an inhibitor of elastase, was upregulated, the result of which would be stability of Elastin in the Extracellular matrix. Elastin is an Extracellular matrix protein that is essential for resilience of the connective tissue of the skin. The cell surface adhesion molecules SELE and ITGA6 were also upregulated in dimethoxytolyl propylresorcinol-treated keratinocytes, and ITGBL1, an integrin-related protein, was downregulated.
These changes together demonstrated the increase in production of Extracellular matrix proteins that dimethoxytolyl propylresorcinol conferred, which was paired with increases in cell adhesion to the extracellular matrix. Differential expression of these genes is listed in Table 9.

TABLE 9
Extracellular matrix genes differentially expressed in
dimethoxytolyl propylresorcinol-treated keratinocytes

| | | | Fold change in |
|---|---|---|---|
| | | Fold change in | dimethoxytolyl |
| Gene Cluster | Gene | Retinol | propylresorcinol |
| Upregulated | | | |
| ECM | HAS2 - hyaluronan synthase 2 | No effect | 5.48 |
| ECM | HAS3 - hyaluronan synthase 3 | No effect | 3.59 |
| ECM | LAMC2 - Laminin subunit gamma 2 | 0.71 | 2.72 |
| ECM | ECM1 - Extracellular matrix protein 1 | No effect | 2.49 |
| ECM | COL1A1 - Collagen type 1 | No effect | 1.89 |
| ECM | LAMA3 - Laminin subunit alpha 3 | No effect | 1.59 |
| ECM | NID2 - Nidogen 2 precursor | 0.79 | 1.45 |
| Elastin genes | PI3 - Peptidase inhibitor 3, elastase | No effect | 10.5 |
| | inhibitor | | |
| Cell adhesion | SELE - E-Selectin | No effect | 99.30 |
| Cell adhesion | ITGA6 - Integrin subunit alpha 6 | 0.59 | 1.44 |
| Downregulated | | | |
| Cell adhesion | ITGBL1 - Integrin subunit beta-like 1 | No effect | 0.68 |

Growth factor and motility signaling was differentially regulated in dimethoxytolyl propylresorcinol-treated keratinocytes compared to Retinol-treated keratinocytes. TGF-β signaling increases expression of extracellular matrix proteins and proteoglycans, and decreases matrix metalloproteinase (MMP) gene expression in the skin (Imfeld D, 2015). We found that dimethoxytolyl propylresorcinol increased expression of the ligand for TGF-β signaling, TGFB1, causing increased signaling of the pathway. IL1A, a cytokine, was also upregulated in dimethoxytolyl propylresorcinol-treated, but not Retinol-treated, keratinocytes. IL-1α is important for inducing wound healing in the skin by causing migration through autocrine signaling to keratinocytes and paracrine signaling to fibroblasts (Chen J D, 1995) (Zheng R, 2019). Differential expression of these genes is listed in Table 10.

TABLE 10
Signal transduction and cytokine genes differentially expressed
in dimethoxytolyl propylresorcinol-treated keratinocytes

| | | | Fold change in |
|---|---|---|---|
| Gene Cluster | | Fold change in | dimethoxytolyl |
| Upregulated | Gene | Retinol | propylresorcinol |
| TGF-β signaling | TGFB1 - Transforming growth factor beta | No effect | 1.30 |
| | 1 | | |
| Cytokine | IL1A - Interleukin 1 alpha | No effect | 6.60 |
| signaling | | | |

PARP15 is a protein-modifying enzyme that affects protein activity. Along with other PARP family members, PARP15 may be involved in transcription regulation through histone modifications, and affect DNA damage repair. SIRT genes are implicated in aging through transcriptional repression by deacetylating histones and transcription factors, though they can deacetylase other protein targets as well. Their activity regulates proliferation and differentiation, and maintains genomic stability (Simon M, 2020) (Dai Y, 2008). SIRT1 gene expression is reduced with age, and SIRT1 activators are being pursued as topical treatments for skin aging due to their protection against UVR-induced aging effects (Bielach-Bazyluk A, 2021). Upregulation of these genes suggests inhibition of skin aging by dimethoxytolyl propylresorcinol may be partly due to transcriptional repression. Differential expression of these genes is listed in Table 11.

TABLE 11
DNA repair and transcriptional regulation genes differentially expressed
in dimethoxytolyl propylresorcinol-treated keratinocytes

| | | | Fold change in |
|---|---|---|---|
| Gene Cluster | | Fold change in | dimethoxytolyl |
| Upregulated | Gene | Retinol | propylresorcinol |
| DNA damage | PARP15 - Poly (ADP-Ribose) | No effect | 98.2 |
| repair/transcription | polymerase family member 15 | | |
| regulation | | | |
| Transcription | SIRT7 - Sirtuin 7 | No effect | 1.41 |
| regulation | | | |
| Transcription | SIRT1 - Sirtuin 1 | No effect | 1.30 |
| regulation | | | |

SERPINs are a family of serine protease inhibitors, which are important for maintaining skin architecture (Cork M J, 2009). SERPINB2 has been shown to have a protective effect on the skin barrier, as knockout mice had reduced barrier function (Schroder W A, 2016). SERPINB2 and SERPINB4 have also been shown to be important for protection from UV irradiation, a major cause of skin aging (Katagiri C, 2006) (Majoros H, 2019). FABP5 is involved in maintenance of skin barrier function by regulating fatty acid synthesis and cell membrane homeostasis. It has been shown that mice deficient in fatty acid binding proteins had altered water barrier function in their skin (Owada Y, 2002). CDK5R1 activates cyclin-dependent kinase 5 (CDK5), a CDK that is primarily expressed in neurons, but has functions in other tissues. CDK5 knockdown mice had impaired epidermal structure, which was found to be due to reduction in Keratin 10 (K10), an epidermal intermediate filament (Dong C, 2017). As an integral tight junction protein, OCLN is involved in maintaining the barrier function of skin cells. These genes affect skin integrity, skin barrier function, and response to damage, processes which are impaired in aging skin (Choi, 2019), are upregulated in dimethoxytolyl propylresorcinol-treated keratinocytes. Differential expression of these genes is listed in Table 12.

TABLE 12
Skin barrier genes differentially expressed in dimethoxytolyl
propylresorcinol-treated keratinocytes

| | | | Fold change in |
|---|---|---|---|
| Gene Cluster | | Fold change in | dimethoxytolyl |
| Upregulated | Gene | Retinol | propylresorcinol |
| Skin barrier | SERPINB4 - Serpin family B member 4 | No effect | 6.37 |
| Skin barrier | SERPINB2 - Serpin family B member 2 | 0.70 | 4.25 |
| Skin barrier | FABP5 - Fatty acid binding protein 5 | No effect | 1.68 |
| Growth/Skin | CDK5R1 - Cyclin-dependent kinase 5 | No effect | 1.54 |
| barrier | activator 1 | | |
| Skin barrier | OCLN - Occludin | No effect | 1.42 |

Oxidative stress caused by intrinsic and extrinsic factors plays a large role in the process of skin aging. UV irradiation, pollution, and other environmental factors are some of the extrinsic causes of oxidative stress. Neutralizing reactive oxygen species in the skin is important for combating skin aging (Rinnerthaler M, 2015). HMOX1 is an enzyme which is upregulated by NRF2, a transcription factor that responds to oxidative stress by upregulating antioxidant genes. AHR is a transcription factor that is activated in response to xenobiotics. It activates genes involved in metabolism, but also can activate an antioxidation program through upregulation of NRF2, SOD1, and other genes (Dietrich, 2016). SOD2 is a reactive oxygen species-neutralizing enzyme, as it directly converts superoxide to hydrogen peroxide and diatomic oxygen. HIF1A is a transcription factor involved in oxygen homeostasis. While it is normally upregulated in oxygen deprivation environments, it also activated in response to a hydrogen peroxide-rich environment, and upregulates antioxidation genes (Li H S, 2019). Through the neutralization of reactive oxygen species, these genes effectively reduce the signs of skin aging. Differential expression of these genes is listed in Table 13.

TABLE 13
Oxidative stress genes differentially expressed in dimethoxytolyl
propylresorcinol-treated keratinocytes

| | | | Fold change in |
|---|---|---|---|
| Gene Cluster | | Fold change in | dimethoxytolyl |
| Upregulated | Gene | Retinol | propylresorcinol |
| Oxidative stress | HMOX1 - Heme oxygenase 1 | No effect | 5.23 |
| Oxidative stress | AHR - Aryl hydrocarbon receptor | No effect | 1.73 |
| Oxidative stress | SOD2 - Superoxide dismutase 2 | No effect | 1.54 |
| Oxidative stress | HIF1A - Hypoxia-inducible factor 1 subunit | No effect | 1.36 |
| | alpha | | |

While NGF is a potent neuronal growth factor, in the skin, it has roles in keratinocyte migration and wound healing (Matsuda H, 1998) (Gostynska N, 2020). Evidence that NGF is downregulated in aged skin suggests that its function is important for maintaining skin function and integrity (Adly M A, 2006). EREG and HBEGF are both ligands for the Epidermal Growth Factor Receptor, EGFR, which regulates many functions of keratinocytes, including proliferation, adhesion and migration, survival, and differentiation. In humans, it's been shown that inhibition of EGFR signaling leads to skin aging phenotypes, including downregulation of hyaluronan synthase genes and upregulation of senescence genes in keratinocytes (Gerber P A, 2016). These growth signaling pathways are critical for skin function and integrity, as evidenced by their reduction in aged skin. Differential expression of these genes is listed in Table 14.

TABLE 14
Growth genes differentially expressed in dimethoxytolyl
propylresorcinol - treated keratinocytes

| | | | Fold change in |
|---|---|---|---|
| | | | Diarylalkane |
| | | | compound with |
| | | | INCI name as |
| Gene Cluster | | Fold change in | Dimethoxytolyl |
| Upregulated | Gene | Retinol | propylresorcinol |
| Growth | NGF - Nerve growth factor | No effect | 113.25 |
| Growth | EREG - Epiregulin | No effect | 9.61 |
| Growth | HBEGF - Heparin-binding EGF-like growth | No effect | 8.61 |
| | factor | | |

Collectively, the changes observed in dimethoxytolyl propylresorcinol-treated keratinocytes indicated the following similarities in differentially regulated skin aging processes to Retinol-treated keratinocytes: Extracellular matrix protein deposition, Lipid dynamics, Cell adhesion, Cell migration, Cytoskeleton remodeling and transport, Neuronal maturation, Immune signaling, and Antioxidation. There were many skin aging processes differentially regulated in dimethoxytolyl propylresorcinol-treated keratinocytes, but not Retinol-treated keratinocytes, including: Extracellular matrix proteins, TGF-β signaling, Transcription regulation, Skin barrier function, Oxidative stress, and Growth. The net effect of these genetic changes describes an anti-aging phenotype in the keratinocytes.

### Example 2: Confirmation of Differential Gene Expression Changes in Dimethoxytolyl Propylresorcinol-Treated Keratinocytes Through Protein Quantification

From Example 1 above, several genes that demonstrated differential regulation that are especially important for skin aging were confirmed through quantification of protein expression. Hacat immortalized human keratinocyte cells were treated with dimethoxytolyl propylresorcinol at 40 μM for 48 hours before being lysed and subjected to Western blot, and cell culture media was subjected to Western blot to quantify protein levels. The dimethoxytolyl propylresorcinol-treated keratinocyte samples were compared to an untreated keratinocyte sample (control). Intracellular proteins of interest are listed in Table 15 and extracellular proteins of interest are listed in Table 16.

TABLE 15
Intracellular genes upregulated in RNA-seq that
had protein levels quantified by Western blot

| Function | Gene | Protein |
|---|---|---|
| ECM | HAS2 | hyaluronan synthase 2 |
| ECM | HAS3 | hyaluronan synthase 3 |
| Cell adhesion | SELE | Selectin E |
| Transcription | SIRT7 | Sirtuin 7 |
| regulation | | |
| Transcription | SIRT1 | Sirtuin 1 |
| regulation | | |
| Skin barrier | SERPINB4 | Serpin family B member 4 |
| Skin barrier | SERPINB2 | Serpin family B member 2 |
| Oxidative stress | SOD2 | Superoxide dismutase 2 |
| Oxidative stress | HIF1A | Hypoxia-inducible factor 1 subunit |
| | | alpha |

TABLE 16
Extracellular genes upregulated in RNA-seq that
had protein levels quantified by Western blot

| Function | Gene | Protein |
|---|---|---|
| ECM | LAMC2 | Laminin subunit gamma 2 |
| ECM | ECM1 | Extracellular matrix protein 1 |
| ECM | COL1A1 | Collagen type 1 alpha 1 chain |
| TGF-β signaling | TGFB1 | Transforming growth factor beta 1 |
| Growth | NGF | Nerve growth factor |
| Growth | EREG | Epiregulin |
| Growth | HBEGF | Heparin-binding EGF-like growth factor |

It was found that LAMC2, ECM1, COL1A1, HAS2, and HAS3 had higher protein levels in the dimethoxytolyl propylresorcinol-treated condition than the control. These are all proteins involved in extracellular matrix composition. These changes imply that anti-wrinkle or anti-aging activity of dimethoxytolyl propylresorcinol is based on increases in extracellular matrix components.

| | Change in dimethoxytolyl propylresorcinol- |
|---|---|
| | treated keratinocytes compared to |
| Protein | Control |
| LAMC2 | +16% |
| ECM1 | +40% |
| COL1A1 | +34% pro-collagen, +27% mature |
| | collagen |
| HAS2 | +14% |
| HAS3 | +17% |

### Example 3: Dimethoxytolyl Propylresorcinol Showed Efficacy Against Fine Lines and Wrinkles in a Human Clinical Trial

To evaluate the anti-oxidant properties of dimethoxytolyl propylresorcinol, a study Pilot Clinical Efficacy Evaluation of a Skin Treatment Product was conducted using dimethoxytolyl propylresorcinol cream at a concentration of 0.2%. The objective of the study was to evaluate if use of the product for 8 weeks would cause a reduction in the appearance of crow's feet fine lines and wrinkles. For this study a panel of 12 subjects ranging from age 35 to 65 was recruited, assessments were conducted at Baseline (BL), Week 2 (W2), Week 4 (W4) and Week 8 (W8) as outlined in Table 17. The study was a single blind, full face, home base study with 4 clinic visits after enrollment.

TABLE 17
Procedures at each clinic visit

| Procedures | Baseline | Week 2 | Week 4 | Week 8 |
|---|---|---|---|---|
| Informed Consent | X | | | |
| Demographics/I/E Criteria | X | | | |
| Visual Assessments | X | X | X | X |
| Instrument Measurements | X | X | X | X |
| Digital photographs Using Visia | X | X | X | X |
| CR ® System. | | | | |
| Skin Replicas Taken | X | X | X | X |
| Study Product Issued | X | X | X | |
| Instructions | X | | | |
| Daily Diary Issued | X | | | |
| Evaluations of Crow's Feet Fine | X | X | X | X |
| Lines/Wrinkles by Trained | | | | |
| Technician | | | | |
| Evaluation of Skin Irritation | X | X | X | X |
| Subject's Questionnaires Issued | | | | X |
| and Collected | | | | |

Included subjects were:
1. Female subjects between the ages of 35-66 (inclusive) in general good health
2. Individuals with a Fine Line/Wrinkle score of “5” (noticeable) or greater at the Crow's Feet area, as assessed by a trained technician and whose wrinkles were evaluated according to the following scale: 0—None, 1-3—Slight, 4-6—Noticeable, 7-9—Very Noticeable.
3. Individuals who could read, understand, and sign the Informed Consent form.
4. Individuals who anticipated the ability to follow the study directions, to participate in the study, to return for all visits and to apply the product as per instructions.
   Excluded subjects were:
1. Women who were pregnant, planning a pregnancy, lactating, and/or nursing a child.
2. Individuals with any visible skin disease that might have interfered with the evaluations.
3. Individuals with sunburn, suntan on the face or planning a vacation with sun-exposure or planning the use of a tanning booth during the course of the study.
4. Individuals engaged in a concurrent research project of a facial product.
5. Individuals taking medications which might have interfered with the test results including the use of steroidal/non-steroidal anti-inflammatory drugs or antihistamines.
6. Individuals who had undergone a laser resurfacing or dermabrasion procedure on the face in the past 2 years or a chemical face peel (deep peel in the past 1 year; superficial peel in the past 2 months).
7. Individuals with acne, active atopic dermatitis/eczema or psoriasis.
8. Individuals who had a surgical “cosmetic” procedure on the face within the past 10 years.
9. Treatment or history of any type of cancer.
10. Individuals who were under treatment for asthma or diabetes.
11. Individuals with a known sensitivity to cosmetics or personal care products.
   At all visits, a trained technician evaluated the appearance of crow's feet, fine lines, and wrinkles based on the following scale: 0—None, 1-3—Slight, 4-6—Noticeable, 7-9—Very Noticeable. The technician also evaluated the face of each subject for irritation. No irritation was found at any point in any of the subjects. Skin replicas were taken on each subject to evaluate fine lines/wrinkles and skin texture as follows: on a glass petri dish, 3 inches of resin (˜3 mL) and 3 drops of the catalyst were mixed thoroughly for about 15-20 seconds. Immediately afterwards, the mixture was placed in CuDerm replica-locating rings that were previously placed on the corner of the eye of each subject. After 4-5 minutes, the rings were removed. Skin replicas obtained were shipped to BioNet, Inc for analysis. At the final visit, subjects were required to complete a questionnaire.

### Results:

Crow's feet, fine lines, and wrinkles were improved after 2, 4, and 8 weeks of dimethoxytolyl propylresorcinol 0.2% cream use, with a clinically significant (≥10%) improvement at week 8 (Table 18).

TABLE 18
Mean crow's feet, fine line, and wrinkle
scores and % change from Baseline

| | Mean Score | Change from Baseline |
|---|---|---|
| Baseline | 6.3 | — |
| Week 2 | 6.2 | −1.6% |
| Week 4 | 6.1 | −3.2% |
| Week 8 | 5.6 | −11.1%* |

*Denotes clinical significance (≥10% improvement)

Over the time course of the study, a higher proportion of subjects experienced an improvement in the appearance of their crow's feet, fine lines, and wrinkles (Table 19).

TABLE 19
Frequency of response of crow's feet, fine lines, and wrinkle
improvement (% of Subjects with improvement from Baseline)

| | % Improving | % No change or worsening |
|---|---|---|
| Week 2 | 8% | 92% |
| Week 4 | 17% | 83% |
| Week 8 | 58% | 42% |

Skin replicas were analyzed using Replica Analysis Methods A and B. Method A measured the luminance along a set of 10 equal length parallel lines running across the replica parallel to the lighting direction. The variations in luminance were treated as indicative of the roughness and analyzed by traditional surface roughness statistics. The following parameters were analyzed using Method A:
1. Rz: the average maximum difference in luminance value for five equal length segments in each of the 10 lines traversing the sample. Maximum optical roughness.
2. Ra: The average deviation of the luminance curve about the mean luminance for the same 10 lines. The average optical roughness.
3. FNum: Number of fine line markers per mm. As lines and creases disappear, F Num decreases.
4. IDL: The integrated developed length of the luminance traces of the 10 scan lines. This is the total length of the luminance lines as portion of the straight-line distance. (i.e., a flat featureless sample has an IDL of 1.000).
   Coarse lines had a statistically significantly 28% reduction in maximum difference in luminance (Rz) and a 32.7% reduction in integrated developed length of the luminance traces 4 weeks after starting dimethoxytolyl propylresorcinol 0.2% cream use, indicating a reduction in surface roughness. There were still trends toward reduction at 8 weeks, but those changes were not statistically significant (Table 20).
   Fine lines had statistically significantly reduced: maximum difference in luminance (Rz), average deviation of the luminance curve (Ra), the number of fine lines per mm (FNum), and the integrated developed length of the luminance traces (IDL) at 2, 4, and 8 weeks of treatment with dimethoxytolyl propylresorcinol 0.2% cream. These consistent and marked reductions in surface roughness gauges, and the notable reduction in the number of fine lines (FNum) indicated clear wrinkle reduction and improvement in skin smoothness (Table 20), even just two weeks after starting dimethoxytolyl propylresorcinol use, and lasting for the course of treatment.

TABLE 20
Changes (and % changes) in Method A parameters
for skin replicas compared to Baseline

| | | | | |
|---|---|---|---|---|
| Week | Rz | Ra | FNUM | IDL |

Coarse Lines

| | | | | |
|---|---|---|---|---|
| 2 | −17.6 | −4.4 | −0.025 | −0.754 |
| | (16.4%) | (19.5%) | (−7.7%) | (17.4%) |
| 4 | −30.0* | −6.7 | −0.033 | −1.418* |
| | (28.0%) | (29.8%) | (10.1%) | (32.7%) |
| 8 | −11.9 | −2.8 | −0.002 | −0.463 |
| | (11.1%) | (12.5%) | (0.6%) | (10.7%) |

Fine Lines

| | | | | |
|---|---|---|---|---|
| 2 | −28.3* | −5.8* | −0.066 | −1.578* |
| | (21.2%) | (22.7%) | (14.0%) | (24.7%) |
| 4 | −23.0* | −4.6* | −0.073* | −1.274* |
| | (17.2%) | (18.0%) | (15.4%) | (19.9%) |
| 8 | −21.6* | −4.0* | −0.069* | −1.323* |
| | (16.2%) | (15.7%) | (14.5%) | (20.7%) |

*Denotes statistical significance (p < 0.05)

The following parameters were analyzed using method B. This method divided the replica image area into 10 equal width bands or sub-areas. Shadow-like features were detected in each of these bands according to their luminance values being less than the detection threshold.
1. Spacing: the mean distance in millimeters between adjacent detected features (i.e., spacing between the midpoints of adjacent shadowy features). Decreases with conversion of deep wrinkles to fine wrinkles (moisturization). Increases with disappearance of wrinkles.
2. Breadth: the average breadth in millimeters of the detected features in millimeters. This parameter is proportional to the depth of the wrinkle producing the shadow. Decreases as wrinkles become shallow.
3. Shadows: percent of the sampled replica area with luminance values less than the detection threshold. This is the relative area of the shadows cast by the wrinkles and the fine lines in the replica. Decreases with smoothing of the skin.
4. Num Wrinkles: the total number of features detected in the 10 bands or subareas used to calculate spacing and breadth. Generally, NumWr decreases with smoothing of the skin (fewer visible features)
   For coarse lines, there were no significant changes in Method B parameters over the 8-week time course of dimethoxytolyl propylresorcinol 0.2% cream use. For fine lines, there were significant 42.3% and 37.5% reductions in shadows cast by wrinkles and fine lines at 2 and 4 weeks, respectively, after the start of the treatment course. At 8 weeks, there was a 30.9% reduction in shadows, which was not statistically significant. The number of wrinkles (NumWr) was statistically significantly reduced at 2, 4, and 8 weeks after starting dimethoxytolyl propylresorcinol use. The number of fine lines was reduced by 37.6% at 2 weeks, 32.8% at 4 weeks, and 29.0% at 8 weeks, demonstrating an immediate and lasting reduction in fine lines conferred by dimethoxytolyl propylresorcinol (Table 21).

TABLE 21
Changes (and % changes) in Method B parameters
for skin replicas compared to Baseline

| | | | | |
|---|---|---|---|---|
| Week | Spacing | Breadth | Shadows | NumWr |

Coarse Lines

| | | | | |
|---|---|---|---|---|
| 2 | 0.808 | 0.012 | −0.8 | −9.9 |
| | (37.6%) | (−5.4%) | (20.9%) | (19.9%) |
| 4 | 0.142 | 0.007 | −0.9 | −12.7 |
| | (6.6%) | (−3.1%) | (25.3%) | (25.4%) |
| 8 | −0.052 | 0.025 | 0.7 | 5.3 |
| | (−2.4%) | (−12%) | (−19%) | (−11%) |

Fine Lines

| | | | | |
|---|---|---|---|---|
| 2 | 0.826 | −0.014 | −1.8* | −26.3* |
| | (47.9%) | (7.5%) | (42.3%) | (37.6%) |
| 4 | 0.358 | −0.001 | −1.6* | −22.9* |
| | (20.8%) | (0.5%) | (37.5%) | (32.8%) |
| 8 | 0.110 | −0.001 | −1.3 | −20.3* |
| | (6.3%) | (0.5%) | (30.9%) | (29.0%) |

*Denotes statistical significance (p < 0.05)

Subjective questionnaire responses were associated with a high rate of subject acceptance of the product. The subjects' perceptions of their own skin were analyzed from the questionnaires and summarized in Table 22. The vast majority of subjects noticed a reduction in the appearance of crow's feet, fine lines, and wrinkles, felt that their skin had a healthier appearance and noticed an improvement in the quality of their skin.

TABLE 22
Summary of subject questionnaire responses at final visit

| | Extremely | |
|---|---|---|
| Since Using the Product, do you | Noticeable to | |
| notice: | Slight Change | No Change |
| The appearance of crow's feet, fine lines, | 83% | 17% |
| and wrinkles has decreased | | |
| Skin has a healthier appearance | 83% | 17% |
| After Using the product: | Agree | Disagree or |
| | | no opinion |
| The overall quality of my skin improved | 67% | 23% |

### Example 4: Dimethoxytolyl Propylresorcinol Showed Efficacy in Improving Skin Brightness and Luminance in an Asian Human Subject Population and Reduced the Appearance of Skin Blotchiness and Age Spots in a Caucasian Human Subject Population

To evaluate the anti-oxidant properties of dimethoxytolyl propylresorcinol, a study Pilot Clinical Efficacy Evaluation of a Skin Treatment Product was conducted using dimethoxytolyl propylresorcinol cream at a concentration of 0.2%.

The objective of the study was to evaluate if use of the product for 8 weeks would cause an improvement in skin brightness/luminescence in an Asian population and reduce the appearance of skin blotchiness/age spots in a Caucasian population. For this study, a panel of 12 subjects (6 6 Asian subjects and 6 Caucasian subjects) ranging from age 35 to 66 was recruited, and assessments were conducted at Baseline (BL), Week 2 (W2), Week 4 (W4) and Week 8 (W8) as outlined in 3. The study was a single blind, full face, home base study with 4 clinic visits after enrollment.

TABLE 23
Procedures at each clinic visit

| Procedures | Baseline | Week 2 | Week 4 | Week 8 |
|---|---|---|---|---|
| Informed Consent | X | | | |
| Demographics/I/E Criteria | X | | | |
| Visual Assessments | X | X | X | X |
| Instrument Measurements | X | X | X | X |
| Digital photographs Using Visia | X | X | X | X |
| CR ® System. | | | | |
| Skin Replicas Taken | X | X | X | X |
| Study Product Issued | X | X | X | |
| Instructions | X | | | |
| Daily Diary Issued | X | | | |
| Evaluations of Brightness/ | X | X | X | X |
| Luminance and Skin | | | | |
| Blotchiness/Age Spots by | | | | |
| Trained Technician | | | | |
| Evaluation of Skin Irritation | X | X | X | X |
| Subject's Questionnaires Issued | | | | X |
| and Collected | | | | |

Included subjects were:
1. Female subjects between the ages of 35-66 (inclusive) in general good health
2. Asian subjects who had skin brightness/luminance score of “5” (moderately dull/sallow appearance) or greater on the face (for qualification only) as assessed by a trained technician and whose brightness/luminance were evaluated according to the following scale: 0—No dullness/sallowness visible, 1-3—Slightly dull/sallow appearance, 4-6—Moderately dull/sallow appearance, 7-9—Severely dull/sallow appearance.
3. Caucasian subjects who had a skin blotchiness/age spot score of “5” (moderate areas of skin blotchiness or age spots) or greater on the face (for qualification only) as assessed by a trained technician and whose blotchiness/age spots were evaluated according to the following scale: 0—No skin blotchiness or age spots visible, 1-3—Slight areas of skin blotchiness or age spots visible, 4-6—Moderate areas of skin blotchiness or age spots visible, 7-9—Severe areas of skin blotchiness or age spots visible.
4. Individuals who could read, understand, and sign the Informed Consent form.
5. Individuals who anticipated the ability to follow the study directions, to participate in the study, to return for all visits and to apply the product as per instructions.
   Excluded subjects were:
1. Women who were pregnant, planning a pregnancy, lactating, and/or nursing a child.
2. Individuals with any visible skin disease that might have interfered with the evaluations.
3. Individuals with sunburn, suntan on the face or planning a vacation with sun-exposure or planning the use of a tanning booth during the course of the study.
4. Individuals engaged in a concurrent research project of a facial product.
5. Individuals taking medications which might have interfered with the test results including the use of steroidal/non-steroidal anti-inflammatory drugs or antihistamines.
6. Individuals who had undergone a laser resurfacing or dermabrasion procedure on the face in the past 2 years or a chemical face peel (deep peel in the past 1 year; superficial peel in the past 2 months).
7. Individuals with acne, active atopic dermatitis/eczema or psoriasis.
8. Individuals who had a surgical “cosmetic” procedure on the face within the past 10 years.
9. Treatment or history of any type of cancer.
10. Individuals who were under treatment for asthma or diabetes.
11. Individuals with a known sensitivity to cosmetics or personal care products.
   At all visits, a trained technician evaluated the face of each subject for irritation. No irritation was found at any point in any of the subjects. Digital images of the face of each subject were taken using the Visia CR® Imaging System. Photographs were taken from the front, right and left views. At the final visit, subjects were required to complete a questionnaire.
   Results: Skin brightness/luminance (Asian subjects): In order to determine any changes in skin brightness/luminance, skin brightness (luminosity) was analyzed by Visia CR®. Facial luminance was a single number calculated based on the uniformity of the lightening of the image. An increase in the facial luminance represented an improvement in overall skin luminance. A decrease represented a worsening. At each visit, Asian subjects rated their skin brightness/luminance according to the following scale: 0—No dullness/sallowness visible, 1-3—Slightly dull/sallow appearance, 4-6—Moderately dull/sallow appearance, 7-9—Severely dull/sallow appearance. Additionally, at the final visit, subjects completed a questionnaire.
   There were no significant changes to brightness/luminance as assessed by the Visia CR® analysis at any of the clinic visits, although the Mean Score for skin brightness/luminance trended toward an increase at 4 and 8 weeks after starting dimethoxytolyl propylresorcinol use compared to Baseline (Table 24), indicating an improvement in skin brightness/luminance.

TABLE 24
Change in brightness/luminance in Asian subjects
as measured by Visia CR ®.

| | Mean Score +/− | | Change from |
|---|---|---|---|
| | S.D. | p Value | Baseline |
| Baseline | 143.10 +/− 10.06 | — | — |
| Week 2 | 141.72 +/− 8.98 | 0.438 | −1.0% |
| Week 4 | 146.30 +/− 9.21 | 0.313 | 2.2% |
| Week 8 | 144.34 +/− 6.33 | 0.563 | 0.9% |

The following table presents the percentage of Asian subjects who showed improvement in the Visia CR® skin brightness/luminance analysis at each visit (Table 25). Skin brightness/luminance was improved in Asian subjects after 4 and 8 weeks of product use, with up to 67% of subjects showing improvement. Despite there not being any significant differences in the Mean Score for skin brightness/luminance, these data demonstrate that there were individual improvements in responders, and that the proportion of responders increased over the treatment course.

TABLE 25
Skin Brightness/Luminance Analysis (Asian Subjects) Frequency
of Response (% of Subjects with Improvement from Baseline).

| | % Improving | % No change or worsening |
|---|---|---|
| Week 2 | 33% | 67% |
| Week 4 | 67% | 33% |
| Week 8 | 67% | 33% |

Self-assessments of Asian subjects at each visit showed an improvement in skin brightness/luminance after 2, 4, and 8 weeks of product use. Notably, after 8 weeks of dimethoxytolyl propylresorcinol use, there was a 13% improvement in self-assessment scores for skin brightness/luminance (Table 26).

TABLE 26
Self-Assessment Scores for Skin Brightness/Luminance -
Asian Subjects

| | Baseline | Week 2 | Week 4 | Week 8 |
|---|---|---|---|---|
| Mean Score | 5.5 | 5.2 | 5.3 | 4.8 |
| % Improvement | — | 6% | 4% | 13% |
| from Baseline | | | | |

Skin Blotchiness/Age Spots (Caucasian subjects): In order to determine any changes in skin blotchiness/age spots, chroma was analyzed by Visia CR®. The degree to which a color is free from being mixed with other colors is a good indication of its chromacity. An increase in the chroma score represented an improvement in skin blotchiness/age spots. A decrease represented a worsening. At each visit, Caucasian subjects rated their skin blotchiness/age spots according to the following scale: 0—No skin blotchiness or age spots visible, 1-3—Slight areas of skin blotchiness or age spots visible, 4-6—Moderate areas of skin blotchiness or age spots visible, 7-9—Severe areas of skin blotchiness or age spots visible. Additionally, at the final visit, subjects completed a questionnaire.
There were no significant changes to skin blotchiness/age spots as assessed by the Visia CR® analysis at any of the clinic visits, although the Mean Score for skin blotchiness/age spots trended toward an increase at all 2, 4, and 8 weeks after starting dimethoxytolyl propylresorcinol use compared to Baseline, indicating an improvement in skin blotchiness/age spots (Table 27).

TABLE 27
Change in skin blotchiness/age spots in Caucasian
subjects as measured by Visia CR ®.

| | Mean Score +/− | | Change from |
|---|---|---|---|
| | S.D. | p Value | Baseline |
| Baseline | 16.67 +/− 2.89 | — | — |
| Week 2 | 17.38 +/− 2.13 | 0.156 | 4.3% |
| Week 4 | 17.90 +/− 1.76 | 0.219 | 7.4% |
| Week 8 | 17.88 +/− 0.98 | 0.219 | 7.3% |

The following table presents the percentage of Caucasian subjects who showed improvement in the Visia CR® skin blotchiness/age spots analysis at each visit (Table 28). Skin blotchiness/age spots were improved in Caucasian subjects after 2, 4, and 8 weeks of product use, with up to 83% of the subjects showing improvement. Despite there not being any significant differences in the Mean Score for skin blotchiness/age spots, these data demonstrate that there were individual improvements in responders, and that a significant proportion of responders retained improvement over the treatment course.

TABLE 28
Skin Blotchiness/Age Spot Analysis (Caucasian Subjects) Frequency
of Response (% of Subjects with Improvement from Baseline).

| | % Improving | % No change or worsening |
|---|---|---|
| Week 2 | 83% | 17% |
| Week 4 | 67% | 33% |
| Week 8 | 67% | 33% |

Self-assessments of Caucasian subjects showed an improvement in skin blotchiness/age spots after 2, 4, and 8 weeks or product use. Notably, after 8 weeks of dimethoxytolyl propylresorcinol use, there was a 37% improvement in self-assessment scores for skin blotchiness/age spots (Table 29).

TABLE 29
Self-Assessment Scores for Skin Blotchiness/Age
Spots - Caucasian Subjects

| | Baseline | Week 2 | Week 4 | Week 8 |
|---|---|---|---|---|
| Mean Score | 6.3 | 4.3 | 4.0 | 4.0 |
| % Improvement | — | 32% | 37% | 37% |
| from Baseline | | | | |

At the final visit, subjects were asked to respond to a questionnaire. Subjective questionnaire responses were associated with a high rate of subject acceptance of the product. The subjects' perceptions of their own skin were analyzed from the questionnaires and summarized in Table 30 and Table 31. The vast majority of Asian subjects noticed an increase in skin brightness/luminance and felt that their skin had a healthier appearance. The majority of Caucasian subjects felt a decrease in the blotchiness of their skin and the appearance of age spots, and felt that their skin had a healthier appearance.

TABLE 30
Responses to the subject questionnaire for Asian subjects

| | Extremely | |
|---|---|---|
| Since using the test product, do you | Noticeable to | |
| notice: | Slight Change | No Change |
| The appearance of skin brightness/ | 83% | 17% |
| luminance has increased: | | |
| Skin has a healthier appearance: | 83% | 17% |
| After using the product: | Agree | Disagree or |
| | | No Opinion |
| The overall quality of my skin improved: | 50% | 50% |
| I would purchase it if it were available on | 17% | 83% |
| the market: | | |
| I would recommend it to a friend: | 17% | 83% |

TABLE 31
Responses to the subject questionnaire for Caucasian subjects

| | Extremely | |
|---|---|---|
| Since using the test product, do you | Noticeable to | |
| notice: | Slight Change | No Change |
| The appearance of skin blotchiness/age | 67% | 33% |
| spots has decreased: | | |
| Skin has a healthier appearance: | 67% | 33% |
| After using the product: | Agree | Disagree or |
| | | No Opinion |
| The overall quality of my skin improved: | 50% | 50% |
| I would purchase it if it were available on | 50% | 50% |
| the market: | | |
| I would recommend it to a friend: | 50% | 50% |

From the above examples, it was demonstrated that dimethoxytolyl propylresorcinol-treated keratinocytes had many differentially expressed genes compared to untreated controls that are involved in aging pathways. Genes expressed in dimethoxytolyl propylresorcinol-treated keratinocytes were compared to genes differentially expressed in Retinol-treated keratinocytes, and there were many common protein locations and biological processes between the treatments, including extracellular region, proteinaceous extracellular matrix, extracellular matrix organization, epidermis development, epithelial cell differentiation, and cellular response to retinoic acid. Specific gene clusters that were similarly regulated in dimethoxytolyl propylresorcinol-treated and Retinol-treated keratinocytes included extracellular matrix genes, cytoskeleton genes, neural cell adhesion, negative regulators of cell growth, immune signaling, and antioxidation. This may imply similar activity between dimethoxytolyl propylresorcinol and Retinol in keratinocytes.

Biological processes differentially expressed in dimethoxytolyl propylresorcinol-treated keratinocytes compared to control, but not in Retinol-treated keratinocytes, included wound healing, keratinocyte differentiation, and hyaluronan biosynthetic process. Specific gene clusters that were differentially regulated in dimethoxytolyl propylresorcinol-treated keratinocytes, but not Retinol-treated keratinocytes, were extracellular matrix genes, cell signaling genes, transcriptional regulation genes, skin barrier genes, oxidative stress genes, and growth factors. A selection of genes that were differentially regulated in dimethoxytolyl propylresorcinol-treated keratinocytes, but not Retinol-treated keratinocytes, were further explored for differential protein expression by Western blot. The changes that were verified included HAS2 and HAS3 upregulation (14% increase and 17% increase, respectively, compared to control keratinocytes), LAMC2 (16% increase), ECM1 (40% increase), and COL1A1 (34% increase in the procollagen and 27% increase in the mature collagen). We could not definitively verify or refute the expression changes for other proteins tested, including SELE, SIRT7, SIRT1, SERPINB4, SERPINB2, SOD2, HIF1A, TGFB1, NGF, EREG, and HBEGF.

The increases in HAS2 and HAS3 indicate increased hyaluronic acid synthesis, and increased capacity for binding water molecules in the extracellular matrix, boosting skin hydration and moisture. The increases in LAMC2, ECM1, and COL1A1 indicate increases in deposition of ECM components. This implies anti-aging potential through reduction and prevention of fine lines and wrinkles by boosting the ECM.

In a clinical trial in which 0.2% dimethoxytolyl propylresorcinol cream was used twice daily for eight weeks, scores for crow's feet, fine lines and wrinkles as assessed by trained technicians were reduced significantly at the end of the study. As evaluated using luminance measurements of skin replicas, dimethoxytolyl propylresorcinol cream reduced the luminance and length of luminance traces of coarse lines after four weeks of use, indicating a reduction in surface roughness. The luminance, number of fine lines, and length of luminance traces for fine lines were significantly reduced even at two weeks of use, and these changes extended to the end of the study at eight weeks, indicating an improvement in skin smoothness. Evaluating fine lines using shadow assessment of skin replicas, dimethoxytolyl propylresorcinol cream significantly reduced shadows cast by wrinkles at just two and four weeks of use. The number of wrinkles and fine lines was also significantly reduced at two weeks and for the duration of the study.

In a clinical assessment of skin luminance, as measured by Visia CR®, in Asian subjects, the Mean Score for brightness/luminance was not changed, but visual assessments by a trained technician indicated improved scores at two weeks of use, with greater improvement at four weeks of use. In parallel, skin blotchiness/age spots were measured by Visia CR® in Caucasian subjects. The Mean Score for blotchiness/age spots was not changed, but again, visual assessments by a trained technician showed improved scores at two weeks of use, with the majority of subjects sustaining improvement throughout the study duration.

These data demonstrate that dimethoxytolyl propylresorcinol cream reduced the appearance of fine lines and wrinkles in a clinical study, with visual scores for luminance and skin blotchiness/age spots also improving over the study duration. The mechanism of action for these improvements may lie in the expression changes of genes involved in extracellular matrix composition, boosting the ECM components, moisturizing the skin, and filling in fine lines and wrinkles.

### REFERENCES

Adly M A, A. H. (2006). Age-associated decrease of the nerve growth factor protein expression in the human skin: Preliminary findings. J Dermatol Sci, 42(3):268-271.
Alvarez-Dolado M, G.-S. J.-Y.-F. (1999). Retinoic acid and 1,25-dihydroxyvitamin D3 inhibit tenascin-C expression in rat glioma C6 cells. J Neurosci Res, 58(2):293-300.
Bielach-Bazyluk A, Z. E.-R. (2021). Sirtuin 1 and Skin: Implications in Intrinsic and Extrinsic Aging—A Systematic Review. Cells, 10,813.
Bukhari S N A, R. N. (2018). Hyaluronic acid, a promising skin rejuvenating biomedicine: A review of recent updates and pre-clinical and clinical investigations on cosmetic and nutricosmetic effects. Int J Biol Macromol, 120(Pt B):1682-1695.
Chen J D, L. J. (1995). Interleukin-1a Stimulates Keratinocyte Migration Through an Epidermal Growth Factor/Transforming Growth Factor-a-Independent Pathway. J Invest Dermatol, 104(5):729-733.
Chen Y C, S. S. (2022). Tyrosinase Inhibitors Derived from Chemical Constituents of Dianella ensifolia. Plants (Basel), 11(16):2142.
Choi, E. H. (2019). Aging of the skin barrier. Clin Dermatol, 37(4):336-345.
Cork M J, D. S.-A. (2009). Epidermal Barrier Dysfunction in Atopic Dermatitis. J Invest Dermatol, 129(8):1892-908.
Dai Y, F. D. (2008). Transcription Regulation by Class III Histone Deacetylases (HDACs)—Sirtuins. Transl Oncogenomics, 3:53-65.
Dietrich, C. (2016). Antioxidant Functions of the Aryl Hydrocarbon Receptor. Stem Cells Int, 7943495.
Dong C, Y. S. (2017). Functional Role of Cyclin-Dependent Kinase 5 in the Regulation of Melanogenesis and Epidermal Structure. Scientific Reports, 7:13783.
Farage M A, M. K. (2007). Structural characteristics of the aging skin: a review. Cutan Ocul Toxicol, 26(4):343-57.
Fogal S, C. M. (2015). Human tyrosinase produced in insect cells: a landmark for the screening of new drugs addressing its activity. Mol Biotechnol, 57(1):45-57.
Fore, J. (2006). A review of skin and the effects of aging on skin structure and function. Ostomy Wound Manage, 52(9):24-35.
Gerber P A, B. B. (2016). Mechanisms of skin aging induced by EGFR inhibitors. Support Care Center, 24(10):4241-4248.
Gostynska N, P. M. (2020). The pleiotropic molecule NGF regulates the in vitro properties of fibroblasts, keratinocytes, and endothelial cells: implications for wound healing. Am J Physiol Cell Physiol, 318(2):360-371.
Gutowska-Owsiak D, P. E. (2020). Addressing Differentiation in Live Human Keratinocytes by Assessment of Membrane Packing Order. Fron Cell Dev Biol, 8:573230.
Haydont V, B. B. (2019). Age-related evolutions of the dermis: Clinical signs, fibroblast and extracellular matrix dynamics. Mech Ageing Dev, 177:150-156.
Imfeld D, J. E. (2015). Activation of TGF-beta: a gateway to skin rejuvenation. Household and Personal Care Today, 10(6):6-11.
Imhof L, L. D. (2021). Topical Over-the-Counter Antiaging Agents: An Update and Systematic Review. Dermatology, 237(2):217-229.
Jia Q, Z. J. (2005). Australia Patent No. AU2005249493B2.
Jia Q, Z. J. (2005). Austria Patent No. AT538778T.
Jia Q, Z. J. (2005). Brazil Patent No. BRPI0510415A.
Jia Q, Z. J. (2005). Canada Patent No. CA2567801C.
Jia Q, Z. J. (2005). China Patent No. CN102210663B.
Jia Q, Z. J. (2005). China Patent No. CN103356514A.
Jia Q, Z. J. (2005). China Patent No. CN109134205B.
Jia Q, Z. J. (2005). China Patent No. CN1993114B.
Jia Q, Z. J. (2005). Europe Patent No. EP1748767B1.
Jia Q, Z. J. (2005). Japan Patent No. JP5128277B2.
Jia Q, Z. J. (2005). Mexico Patent No. MXPA06013705A.
Jia Q, Z. J. (2005). Poland Patent No. PL1748767T3.
Jia Q, Z. J. (2005). Russia Patent No. RU2006140961A.
Jia Q, Z. J. (2005). Russia Patent No. RU2466981C1.
Jia Q, Z. J. (2005). South Africa Patent No. ZA200609927B.
Jia Q, Z. J. (2005). South Korea Patent No. KR101236527B1.
Jia Q, Z. J. (2005). South Korea Patent No. KR20120117948A.
Jia Q, Z. J. (2005). Spain Patent No. ES2384506T3.
Jia Q, Z. J. (2005). United States Patent No. US20050267047A1.
Jia Q, Z. J. (2005). WIPO Patent No. WO2005117849A1.
Jia Q, Z. J. (2007). Hong Kong Patent No. HK1095536A1.
Jia Q, Z. J. (2008). U.S. Pat. No. 7,767,661B2.
Jia Q, Z. J. (2010). U.S. Pat. No. 8,592,488B2.
Jia Q, Z. J. (2012). U.S. Pat. No. 8,729,136B2.
Jia Q, Z. J. (2014). U.S. Pat. No. 9,126,913B2.
Jia Q, Z. J. (2015). U.S. Pat. No. 10,548,825B2.
Jia Q, Z. J. (2020). U.S. Pat. No. 10,857,082B2.
Jia Q, Z. J. (2020). United States Patent No. US20210085580A1.
Katagiri C, N. J. (2006). Serpin squamous cell carcinoma antigen inhibits UV-induced apoptosis via suppression of c-JUN NH2-terminal kinase. J Cell Biol, 172(7):983-990.
Krzeminska A, K. N. (2022). Theoretical Studies of Cyanophycin Dipeptides as Inhibitors of Tyrosinases. Int J Mol Sci, 23(6):3335.
Langton A K, H. P. (2016). The impact of intrinsic ageing on the protein composition of the dermal-epidermal junction. Mech Ageing Dev, 156:14-16.
Li H S, Z. Y. (2019). HIF-1a protects against oxidative stress by directly targeting mitochondria. Redox Biol, 25:101109.
Majoros H, U. Z. (2019). SerpinB2 is involved in cellular response upon UV irradiation. Scientific Reports, 9:2753.
Masaki, H. (2010). Role of antioxidants in the skin: anti-aging effects. J Dermatol Sci, 58(2):85-90.
Matsuda H, K. H. (1998). Role of Nerve Growth Factor in Cutaneous Wound Healing: Accelerating Effects in Normal and Healing-impaired Diabetic Mice. J Exp Med, 187(3):297-306.
Michalak M, P. M. (2021). Bioactive Compounds for Skin Health: A Review. Nutrients, 13(1):203.
Mulla T, P. S. (2019). The Binding Affinity of Small Molecues with Yam Tyrosinase (Catechol Oxidase): A Biophysical Study. Biochem Res Int, 2019:8284968.
Nesterov A, Z. J. (2008). 1-(2,4-dihydroxyphenyl)-3-(2,4-dimethoxy-3-methylphenyl)propane, a novel tyrosinase inhibitor with strong depigmenting effects. Chem Pharm Bull, 56(9):1292-6.
Niki Y, Y. M. (2011). 1-(2,4-Dihydroxyphenyl)-3-(2,4-dimethoxy-3-methylpheny)propane inhibits melanin synthesis by dual mechanisms. J Dermatol Sci, 63(2):115-21.
Owada Y, T. H. (2002). Altered water barrier function in epidermal-type fatty acid binding protein-deficient mice. J invest Dermatol, 118(3):430-5.
Placines C, C.-L. V.-M. (2020). Phenolic Profile, Toxicity, Enzyme Inhibition, In Silico Studies, and Antioxidant Properties of Cakile maritima Scop. (Brassicaceae) from Southern Portugal. Plants (Basel), 9(2):142.
Ramia de Cap M, P. X. (2021). Exogenous ochronosis associated with dimethoxytolyl propylresorcinol (UP302). J Dermatol, 48(7):e312-e313.
Rinnerthaler M, B. J. (2015). Oxidative Stress in Aging Human Skin. Biomolecules, 5(2):545-589.
Schroder W A, A. I. (2016). SerpinB2 Deficiency Results in a Stratum Corneum Defect and Increased Sensitivity to Topically Applied Inflammatory Agents. Am J Pathol, 186(6):1511-23.
Simon M, E. S. (2020). A hairy tail: SIRT7 safeguards skin stem cells during aging. EMBO J, 39(18):e106294.
Zasada M, B. (2019). Retinoids: active molecules influencing skin structure formation in cosmetic and dermatological treatments. Postepy Dermatol Alergol, 36(4):392-397.
Zhang S Q, Z. L. (2011). Quantification of a novel natural antioxidant (UP302) in rat plasma using ultra-high performance liquid chromatography-tandem mass spectrometry. J Chromatogr B Analyt Technol Biomed Life Sci, 879(31):3763-6.
Zheng R, L. W. (2019). Keratinocyte Integrin a3B1 Promotes Secretion of IL-1a to Effect Paracrine Regulation of Fibroblast Gene Expression and Differentiation. J Invest Dermatol, 139(9):2029-2083.

1. A diarylalkane compound for use in anti-aging, wrinkle reduction, and extracellular matrix-boosting.

2. The compound of claim 1, wherein the diarylalkane compound comprises dimethoxytolyl propylresorcinol.

3. The compound according to claim 1 for use to reduce the number of fine lines and wrinkles on skin.

4. The compound according to claim 1 for use to reduce the appearance of fine lines and wrinkles on skin.

5. The compound according to claim 1 for use to increase skin smoothness.

6. The compound according to claim 1 for use to increase radiant/luminous appearance of skin.

7. The compound according to claim 1 for use to reduce the number of coarse lines on skin.

8. The compound according to claim 1 for use to reduce the appearance of coarse lines on skin.

9. The compound according to claim 1 for use to increase hyaluronic acid synthesis and skin hydration.

10. The compound according to claim 1 for use to increase production of components of the extracellular matrix.

11. The compound of claim 1, wherein an anti-aging benefit is achieved by regulating expression changes of genes of skin keratinocytes involved in extracellular matrix composition, boosting the ECM components, moisturizing the skin, and filling in fine lines and wrinkles.

12. The compound of claim 2, wherein an anti-aging benefit is achieved by regulating expression changes of genes of skin keratinocytes involved in extracellular matrix composition, boosting the ECM components, moisturizing the skin, and filling in fine lines and wrinkles.

13. The compound of claim 1, wherein at least one gene associated with functions as wound healing, cytoskeletal regulation, antioxidation, immune signaling, cell growth, cell signaling, DNA repair, transcriptional regulation, and skin barrier function is upregulated.

14. The compound of claim 2, wherein at least one gene associated with functions as wound healing, cytoskeletal regulation, antioxidation, immune signaling, cell growth, cell signaling, DNA repair, transcriptional regulation, and skin barrier function is upregulated.

15. The compound of claim 1, wherein at least one anti-aging benefit is achieved by regulating protein expressions of HAS2, HAS3, LAMC2, ECM1 (40% increase), COL1A1, SELE, SIRT7, SIRT1, SERPINB4, SERPINB2, SOD2, HIF1A, TGFB1, NGF, EREG, and HBEGF.

16. The compound of claim 2, wherein at least one anti-aging benefit is achieved by regulating protein expressions of HAS2, HAS3, LAMC2, ECM1 (40% increase), COL1A1, SELE, SIRT7, SIRT1, SERPINB4, SERPINB2, SOD2, HIF1A, TGFB1, NGF, EREG, and HBEGF.

17. The compound of claim 1, wherein the compound is 1-(3-methyl-2,4-dimethoxyphenyl)-3-(2′,4′-dihydroxyphenyl)-propane, or 1-(3-methyl-2,4-dimethoxyphenyl)-3-(2′,5′-dihydroxyphenyl)-propane.

18. The compound of claim 2, wherein the compound is 1-(3-methyl-2,4-dimethoxyphenyl)-3-(2′,4′-dihydroxyphenyl)-propane, or 1-(3-methyl-2,4-dimethoxyphenyl)-3-(2′,5′-dihydroxyphenyl)-propane.

19. The compound of claim 1, wherein the compound is extracted, enriched, and purified from Dianella ensifolia.

20. The compound of claim 2, wherein the compound is extracted, enriched, and purified from Dianella ensifolia.

21. The compound of claim 1, wherein the compound is synthesized and purified.

22. The compound of claim 2, wherein the compound is synthesized and purified.

23. The compound of claim 1, wherein the compound is biosynthesized from plant tissues or fungi tissues, stem cells and transgenic microbials and synthetically modified by isolated or expressed enzymes.

24. The compound of claim 2, wherein the compound is biosynthesized from plant tissues or fungi tissues, stem cells and transgenic microbials and synthetically modified by isolated or expressed enzymes.

25. The compound of claim 1, wherein the compound is formulated in a carrier and may comprise a solution, emulsion, cream, lotion, ointment, or gel comprising one or more of the following: petrolatum, lanolin, polyethylene glycols, bee wax, mineral oil, diluents such as water and alcohol, and emulsifiers and stabilizers.

26. The compound of claim 2, wherein the compound is formulated in a carrier and may comprise a solution, emulsion, cream, lotion, ointment, or gel comprising one or more of the following: petrolatum, lanolin, polyethylene glycols, bee wax, mineral oil, diluents such as water and alcohol, and emulsifiers and stabilizers.

27. A composition comprising the compound of claim 1, wherein the compound is in the composition in an amount between 0.001-2%.

28. A composition comprising the compound of claim 2, wherein the compound is in the composition in an amount between 0.001-2%.

29. A composition comprising the compound of claim 1, wherein the compound is in the composition in an amount of 0.2%.

30. A composition comprising the compound of claim 2, wherein the compound is in the composition in an amount of 0.2%.
